(19) United States
(12) Patent Application Publication
(10) Pub. No.: US 2026/0240431 A1
(43) Pub. Date: Aug. 20, 2026

(54) APPARATUS FOR EXAMINING THE RETINA OF AN EYE

(71) Applicant: Heidelberg Engineering GmbH, Heidelberg (DE)

(72) Inventors: Michael STENDER, Lübeck (DE); Andreas FRITZ-BOUTELEUX, Lübeck (DE)

(73) Assignee: Heidelberg Engineering GmbH, Heidelberg (DE)

(21) Appl. No.: 19/470,859

(22) PCT Filed: May 15, 2024
(86) PCT No.: PCT/EP2024/063355

§ 371 Date: Sep. 30, 2025

(30) Foreign Application Priority Data
Jun. 15, 2023 (DE) 10 2023 115 655.1

(51) Int. Cl. A61B 3/10; A61B 3/13

(52) U.S. Cl. CPC A61B 3/102; A61B 3/13

(57) ABSTRACT

The invention relates to an apparatus for examining the retina (10) of an eye (7), comprising an OCT device (2) for recording OCT images (14) by means of optical coherence tomography (OCT) and a further device (4) for recording further images (13) of the retina (10), characterized in that a data processing unit (18) uniquely assigns an OCT image (14) to at least one further image (13) and uses structures (17) of the retina (10) captured both in the OCT image (14) and the further image (13) as calibration targets for the OCT device (2) and the further device (4).

The invention relates to a device according to the preamble of claim 1.

The term “optical coherence tomography” (usually abbreviated to OCT) is understood to be an imaging method. With this method, two-dimensional and three-dimensional images can be obtained from light-scattering structures. In this method, light with a specific bandwidth is typically split into two partial beams in a beam splitter. The first partial beam is incident on the sample or object to be examined, the second partial beam passes through a reference section. The light reflected by the sample or object interferes with the reference beam. Signals from the interference can be used to examine the sample with a depth resolution, i.e. in the depth of the optical axis of the first partial beam, by means of what are known as A-scans. In addition, it is possible to also scan the surface of the sample or to scan the sample laterally with the first partial beam in order to obtain OCT images. A B-scan is composed of multiple A-scans.

In the field of intraoperative OCT, it is of the utmost importance that an OCT scan position for obtaining an OCT image and a microscope image in particular can be unambiguously assigned to each other. This assignment is realized as standard by calibration targets or calibration markers, which are recognizable in both modalities or image categories.

Both image categories visualize the calibration target, and a suitable coordinate transformation is determined, the latter governing the assignment of scan points for an OCT image to pixels for a microscope image.

This calibration assumes that no additional optical elements that have different effects on the two image categories are present in a system. However, this is often the case in the field of retinal imaging.

To image the retina of an eye, an additional optics unit (e.g. Oculus BIOM (brand name)) which realizes imaging at infinity is swiveled into a beam path. The refractive elements of the eye to be examined then focus beams on the retina to the best possible extent by way of the eye optics.

The non-collinearity of an OCT beam or OCT beam path with respect to the imaging beam path of a microscope causes an offset of pixels in the two image categories. This offset is different as a result of individual refraction in different eyes. A similar influence is exerted by the surgery-specific (operation-specific) configuration of an additional optics unit, for example the focus position of a loupe in the Oculus BIOM system, and the relative eye position.

Furthermore, individual chromatic aberrations of an eye shift the imaging points on the retina, even in the case of collinearity of the aforementioned beam paths. For these reasons, an imaging system cannot be calibrated in a general way. Without individual calibration, misjudgments may occur during an operation (surgery).

The problem addressed by the invention is therefore that of specifying an apparatus that allows the generation of both OCT images and further images that image or capture the same region to be examined as accurately as possible and without optical offset relative to each other.

The present invention solves the aforementioned problem by way of the features of claim 1.

According to the invention, an apparatus for examining the retina of an eye comprises an OCT device for recording OCT images by means of optical coherence tomography and a further device for recording images of the retina. Further, according to the invention, a data processing unit uniquely assigns an OCT image to at least one further image and uses structures of the retina, which are captured in both the OCT image and the further image, as calibration targets for the OCT device and the further device.

According to the invention, it has first been recognized that especially during an operation there is a need for the realization of an individual, preferably continuous, calibration of an OCT scan position in relation to a further image, for example a microscope image. It has subsequently been recognized that an individual calibration is possible by using the aforementioned structures, and said individual calibration can overwrite a general calibration for at least a moment. This can ensure that the scan position and the further image are displayed correctly. The images capture or image the same region to be examined, as accurately as possible and without optical offset relative to each other.

According to the invention, it has been recognized in particular that, up to this point, the already known intraoperative OCT systems have not taken sufficient account of the following problem, specifically the problem of the scan position in the retinal region not being uniquely assigned to the further image, specifically a microscope image. Using the technical solution described herein, it is possible to perform OCT scans at precisely defined positions.

Against this background, the data processing unit could bring the scan coordinates of the OCT image into correspondence with the corresponding pixel coordinates of the further image on the basis of the positions of the structures in the OCT image and in the further image. Characteristic structures can thus be used precisely as calibration targets.

The data processing unit could map the scan coordinates of the OCT image and the pixel coordinates of the further image onto each other by way of a coordinate transformation. This allows different images representing a region to be overlaid or placed side-by-side and analyzed.

The OCT image could be configured as an en-face image, which is generated from an OCT volume recording of the tissue of the eye. Especially during the operation, an en-face OCT image, i.e. an OCT slice image, allows registration of this recording with the further images of the further device. This maps the pixels and scan coordinates onto each other. An individual calibration can be performed particularly effectively in this way. This individual calibration then overwrites a general calibration for a moment. This can ensure that the scan position and a further image are displayed correctly.

The OCT volume recording could be stored in a data memory. This allows access to the OCT volume recording at all times. An en-face image provides cross-sectional views in the tissue. Recording an en-face image or an en-face recording takes a significant amount of time, depending on the size. It is therefore advantageous to use a particularly fast OCT system. A-scan rates in the MHz range, in particular, are preferred in this context. Particularly fast scanners and fast scanner control are also preferred. Closed scan patterns, such as a spiral, can further increase the efficiency of the scans, and so a recalibration can optionally be performed quasi-continuously at certain intervals.

The data processing unit and/or a control unit could bring the scan position or capture position of the OCT image on the retina into correspondence with that of the further image, preferably simultaneously, such that an optical offset of the beam paths from the OCT device and the further device by virtue of additional optical components and/or refractive behavior of the examined eye is compensated. Thus, optical-mechanical influences can be compensated for electronically and hence quickly.

The data processing unit and/or the control unit could bring the scan position or capture position of the OCT image on the retina into correspondence with that of the further image in periodically repeating fashion. This allows a calibration to be adapted to changing recording conditions. An operation can be performed reliably over a relatively long period of time because the apparatus independently recalibrates itself automatically and/or at predefined time intervals.

The OCT image might not be visualized. In an alternative to that or in addition, the OCT image could be configured as an image data set. This makes it possible to visualize only the further image for analysis and merely use the OCT image data for calibration purposes.

The further image could be configured as a fundus image. The further device could therefore comprise an SLO (scanning laser ophthalmoscope) or a fundus camera.

The further device could be configured as a microscope, in particular as a surgical microscope, and the further image as a microscope image. This makes it possible to record greatly magnified images of regions of the retina.

Posterior segment imaging by way of OCT in a surgical microscope is an important augmentation for existing OCT apparatuses. In ophthalmic surgery, posterior segment procedures that require high positioning accuracy play a major role.

The apparatus described herein could also be used for high-precision subretinal injection, especially in gene therapy for the treatment of retinal diseases. In this respect, it is essential to align the OCT image or OCT slice image with the injection needle in order to be able to observe the injection volume. This can reduce the administered volume of the very expensive administered drug, leading to significant cost savings.

An arrangement could comprise an apparatus of the type described herein and additional optical components for examining the retina of an eye.

In the drawing:

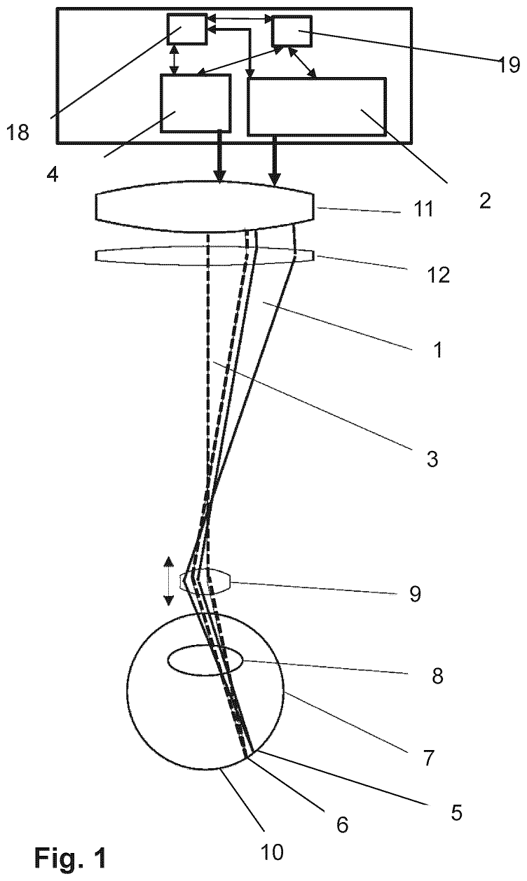

FIG. 1 shows a basic illustration of the beam paths of an OCT device and a microscope, and

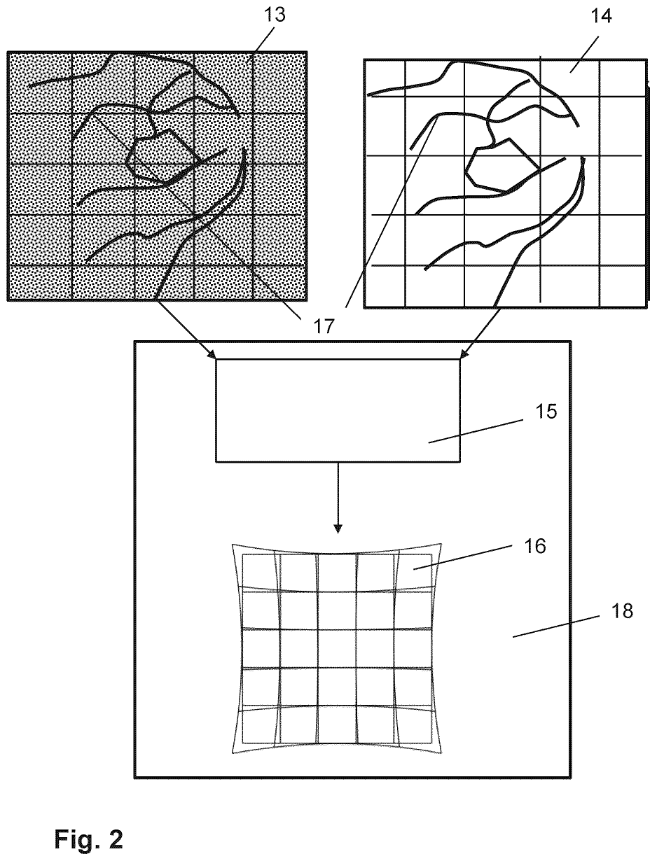

FIG. 2 shows, in the upper view, a microscope image on the left and an en-face image recorded by an OCT device on the right, said images being registered with each other by a method step schematically depicted in the middle and being transformed by a schematically depicted method step.

FIG. 1 shows a non-collinearity of an OCT beam path 1 of an OCT device 2 relative to the imaging beam path 3 of a further device 4, specifically a microscope in this case, said non-collinearity causing an offset of the pixels 5, 6 in the image categories of OCT image 14 and further image 13, specifically microscope image.

This offset is caused inter alia by the individual refraction of the eye 7 to be examined, in particular by its eye lens 8.

A further influence is exerted by the operation-specific configuration of an additional optics unit, specifically the focus position of a loupe 9, and the relative eye position.

Furthermore, the individual chromatic aberrations of the eye 7 shift the imaging points 5, 6 on the retina 10, even in the case of assumed collinearity of the two beam paths 1,3.

The apparatus, which comprises the OCT device 2 and the microscope, is assigned a front lens 11 and a reducing lens 12.

In this respect, FIG. 1 schematically shows an apparatus for examining the retina 10 of an eye 7, comprising an OCT device 2 for recording OCT images 14 by means of optical coherence tomography (OCT) and a further device 4 for recording further images 13 of the retina 10.

Such images are shown in FIG. 2. The further device 4 is configured as a microscope, in particular as a surgical microscope, and the further image 13 is configured as a microscope image.

FIG. 2 schematically shows that in addition to continuous microscope images 13 a single OCT volume is recorded, from which an OCT image 14, specifically an en-face image, is generated. The OCT image 14 is configured as an en-face image, which is generated from an OCT volume recording of the tissue of the eye 7. The OCT volume recording is stored in a data memory. This OCT image 14 does not necessarily serve for visualization but preferably for, or only for, registration 15. The OCT image 14 is therefore preferably not visualized but configured as an image data set.

In the en-face view, some of the same structures 17 appear as in the microscope image 13. This enables the use thereof for registration 15. Using known registration methods, a transformation of the images 13, 14 can be found and applied. This transformation aligns the structures 17 in the two image categories.

This transformation is not only valid for the en-face image but it generally describes the transformation of the scan coordinates to the corresponding pixel coordinates of the microscope. This opens up possibilities, such as the correct positioning of a B-scan as it should be performed in the microscope image or on the basis of the microscope image.

FIGS. 1 and 2 therefore schematically show that a data processing unit 18 uniquely assigns an OCT image 14 to at least one image 13 and uses structures 17 of the retina 10, which are captured in both the OCT image 14 and the image 13, as calibration targets for the OCT device 2 and the further device 4.

The data processing unit 18 brings the scan coordinates of the OCT image 14 into correspondence with the corresponding pixel coordinates of the image 13 on the basis of the positions of the structures 17 in the images 13, 14.

The data processing unit 18 maps the scan coordinates of the OCT image 14 and the pixel coordinates of the image 13 onto each other by way of a coordinate transformation.

The data processing unit 18 and a control unit 19 bring the scan position or capture position of the OCT image 14 on the retina 10 into correspondence with that of the image 13, preferably simultaneously, such that an optical offset of the beam paths 1, 3 from the OCT device 2 and the further device 4 by virtue of additional optical components and/or refractive behavior of the examined eye 7 is compensated.

The data processing unit 18 and the control unit 19 bring the scan position or capture position of the OCT image 14 on the retina 10 into correspondence with that of the image 13 in periodically repeating fashion in order to adapt a calibration to changing recording conditions.

FIG. 1 also shows an arrangement, comprising an apparatus of the type described herein and additional optical components for examining the retina 10 of an eye 7. The additional components comprise a loupe 9, a front lens 11 and a reducing lens 12.

### List of Reference Signs

1 OCT beam path
2 OCT device
3 Imaging beam path of 4
4 Further device, specifically a microscope in this case
5 Pixel of 1
6 Pixel of 3
7 Eye
8 Eye lens
9 Loupe
10 Retina
11 Front lens
12 Reducing lens
13 Further image, specifically a microscope image in this case
14 OCT image or en-face image
15 Registration
16 Transformation
17 Structures of 10

1. An apparatus for examining the retina (10) of an eye (7), comprising an OCT device (2) for recording OCT images (14) by means of optical coherence tomography (OCT) and a further device (4) for recording further images (13) of the retina (10),
wherein a data processing unit (18) uniquely assigns an OCT image (14) to at least one further image (13) and uses structures (17) of the retina (10), which are captured in both the OCT image (14) and the further image (13), as calibration targets for the OCT device (2) and the further device (4).

2. The apparatus as claimed in claim 1, wherein the data processing unit (18) brings the scan coordinates of the OCT image (14) into correspondence with the corresponding pixel coordinates of the further image (13) on the basis of the positions of the structures (17) in the images (13, 14).

3. The apparatus as claimed in claim 2, wherein the data processing unit (18) maps the scan coordinates of the OCT image (14) and the pixel coordinates of the further image (13) onto each other by way of a coordinate transformation.

4. The apparatus as claimed in claim 1, wherein the OCT image (14) is configured as an en-face image, which is generated from an OCT volume recording of the tissue of the eye (7).

5. The apparatus as claimed in claim 4, wherein the OCT volume recording is stored in a data memory.

6. The apparatus as claimed in claim 1, wherein the data processing unit (18) and/or a control unit (19) bring or brings the scan position or capture position of the OCT image (14) on the retina (10) into correspondence with that of the further image (13), preferably simultaneously, such that an optical offset of the beam paths (1, 3) from the OCT device (2) and the further device (4) by virtue of additional optical components and/or refractive behavior of the examined eye (7) is compensated.

7. The apparatus as claimed in claim 6, wherein the data processing unit (18) and/or a control unit (19) bring or brings the scan position or capture position of the OCT image (14) on the retina (10) into correspondence with that of the further image (13) in periodically repeating fashion in order to adapt a calibration to changing recording conditions.

8. The apparatus as claimed in claim 5, wherein the OCT image (14) is not visualized and/or is configured as an image data set.

9. The apparatus as claimed in claim 7, wherein the further device (4) is configured as a microscope and the further image (13) is configured as a microscope image.

10. An arrangement comprising an apparatus as claimed in claim and additional optical components for examining
